(19) United States
(12) Patent Application Publication
(10) Pub. No.: US 2023/0398233 A1
(43) Pub. Date: Dec. 14, 2023

(54) OCULAR ANTIBODY-DRUG CONJUGATES

(71) Applicant: ADS THERAPEUTICS LLC, Irvine, CA (US)

(72) Inventors: Jinsong NI, Irvine, CA (US); Rong YANG, Irvine, CA (US); Wenkui Ken FANG, Irvine, CA (US)

(21) Appl. No.: 18/034,710

(22) PCT Filed: Nov. 3, 2021
(86) PCT No.: PCT/US2021/057844

§ 371 Date: Apr. 30, 2023

Related U.S. Application Data
(60) Provisional application No. 63/108,990, filed on Nov. 3, 2020.

(51) Int. Cl. A61K 47/68; C07K 16/22; A61K 31/573; A61K 9/00

(52) U.S. Cl. CPC A61K 47/6889; C07K 16/22; A61K 47/6803; A61K 47/6845; A61K 31/573; A61K 9/0048; C07K 2317/94

(57) ABSTRACT

An ocular antibody-drug conjugate compound is provided. The compound includes an antibody, the antibody being a classic antibody or a modified biologic molecule that blocks a first target in the subject; a small molecule drug including an alpha agonist or an anti-inflammatory small molecule selected from a steroid, a NSAID, that regulates a second or more target in the subject; and a linker linking the antibody and the small molecule drug. Methods of treating an ocular disease with the antibody drug conjugate compound are also provided. The linker is hydrolyzed in the subject over a certain time so that both the antibody and the steroid exert their functions simultaneously.

The present invention claims priority to U.S. Provisional Patent Application No. 63/108,990, filed Nov. 3, 2020, which is incorporated by reference for all purposes as if fully set forth herein.

### TECHNICAL FIELD

The present invention relates to antibody-drug conjugate compounds and methods of using the antibody-drug conjugate compounds. For example, ocular antibody-drug conjugate compounds are provided herein, as well as methods that can be used to treat a subject having an ocular disorder, such as an eye disease.

### BACKGROUND OF THE INVENTION

Anti-angiogenesis strategies are effective treatments for ocular neovascular diseases such as exudative AMD (also known as wet AMD). Several anti-VEGF antibodies or engineered biologics are on the market for AMD. Despite the effectiveness of these drugs, improvements for this and other ocular diseases are needed. For example, the treatment of anti-VEGF resistant patients by exploring new mechanism of actions; the need to reduce treatment injection frequency or different ways to deliver the drugs. The disclosure provides a novel way to increase the effectiveness for AMD and other ocular neovascular diseases.

### SUMMARY OF THE INVENTION

The present disclosure provides compounds and methods for enhanced efficacy in treating an ocular disease in a subject. Ocular antibody-drug conjugate compounds are provided herein, comprising: an antibody, the antibody being a classic antibody or a modified biologic molecule that reduces neovascularization—the first target of the disease in the subject, a small molecule drug, that modulates the second target of the disease in the subject; and a linker between the antibody and the drug to form a conjugate compound for the treatment of ocular diseases. The linker, covalently attached between the antibody and the small molecule, is hydrolyzed in certain tissues such as vitreous humor of a subject over a certain period of time so that both the antibody and the small molecule can be dissociated to both exert their functions in the subject. In some embodiments, the antibody can be anti-VEGF antibody and the small molecule can be anti-inflammatory drugs. In some embodiments, both the anti-VEGF antibody and the anti-inflammatory small molecule, upon hydrolysis and release in certain tissues such as vitreous humor, can exert their functions in a subject simultaneously. In some embodiments, the conjugate compound can confer better efficacy than either the antibody or the steroid alone, or can exhibit a synergism between the two. In some embodiments, the conjugate compound can provide effective treatment to patients that are non-responders or poor-responders to anti-VEGF antibody. In some embodiments, a method is provided for treating an ocular disease in a subject, comprising delivering the compound or conjugate to the subject, wherein the linker is hydrolyzed in the subject over time such that both the antibody and the small molecule steroid exert their functions in the subject. In some embodiments, the conjugate compound can provide prolonged effective treatment to patients to reduce adverse effects due to frequent antibody intravitreal injections.

In one embodiment, the present application discloses a compound that includes: an antibody or engineered biologic molecule that blocks VEGF, VEGFR, PDGF, PDGFR, FGF, or FGFR; a small molecule drug, the small molecular drug being an adrenergic receptor alpha agonist or an anti-inflammatory small molecule, the anti-inflammatory small molecule being a steroid or a non-steroid anti-inflammatory drug (NSAID); and a linker that links the small molecule drug to the antibody or engineered biologic molecule. The linker comprises a bond that can be hydrolyzed in ocular tissue in a controlled release fashion.

In another embodiment, the antibody is an anti-VEGF-A antibody.

In another embodiment, the antibody is selected from group consisting of bevacizumab, ranibizumab, brolucizumab, aflibercept, and conbercept, preferably, the antibody is bevacizumab.

In another embodiment, the antibody is pegylated to include a polyethylene glycol (PEG) moiety that is either linear or branched.

In another embodiment, the PEG moiety is —(CH₂—CH₂—O—)ₙ—, and n is 5-30, preferably, n is 10-15.

In another embodiment, the linker links the small molecule drug via the PEG moiety to the antibody or engineered biologic molecule.

In another embodiment, the linker comprises an ester, an amide, a carbamate, a carbonate, an imine, an ether, a phosphate, a hydrazone, an acetal, or a hydrozone bond, preferably, the linker comprises an ester bond.

In another embodiment, the linker is

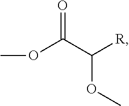

and R is H, —C₁₋₁₈ alkyl, -aryl, heteroaryl, —C₁₋₁₈ alkylaryl, or -alkylheteroaryl, preferably, R is H, methyl, ethyl, propyl, isopropyl, t-butyl, phenyl, or benzyl.

In another embodiment, the steroid is selected from group consisting of dexamethasone, betamethasone, prednisone, prednisolone, triamcinolone, tethylprednisolone, hydrocortisone, cortisone acetate, fludrocortisone, and aldosterone, preferably, the steroid is dexamethasone.

In another embodiment, the NSAID is selected from the group consisting of aspirin, celecoxib, bromfenac, diclofenac, diflunisal, etodolac, ibuprofen, indomethacin, ketoprofen, ketorolac, nabumetone, naproxen, oxaprozin, piroxicam, salsalate, sulindac, and tolmetin, preferably, the NSAID is bromfenac.

In another embodiment, the adrenergic receptor alpha agonist is selected from the group consisting of apraclonidine, mivaZerol, clonidine, brimonidine, alpha methyl dopa, guanfacine, dexemeditomidine, (+)-(S)-4-1-(2,3-dimethyl-phenyl)-ethyl-1,3-dihydro-imidazole-2-thione, 1-(imidazolidin-2-yl)iminolindazole, methoxamine, phenylephrine, tizanidine, xylazine, guanabenz, and amitraz, preferably, the adrenergic receptor alpha agonist is brimonidine.

In another embodiment, the compound includes: bevacizumab; dexamethasone; a PEG moiety; and a linker. The PEG moiety is —(CH₂—CH₂—O—)ₙ—, n is 5-30; the linker is

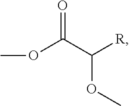

and R is H, methyl, ethyl, propyl, isopropyl, t-butyl, phenyl, or benzyl; and the linker links dexamethasone via the PEG moiety to bevacizumab.

In another embodiment, the hydrolysis of the linker in ocular tissues is controlled and a time for linker hydrolysis of half of the compound is 1-60 minutes, 1-24 hours, 1-5 days, or 1-30 days, preferably, 1-5 days.

In another embodiment, the present application includes a method of treating an ocular disease in a subject. The method includes delivering the compound of the present application to an eye of the subject.

In another embodiment, the method further includes allowing the linker to hydrolyze in one or more ocular tissues of the eye of the subject over time. Both the antibody and the small molecule drug exert their functions in the subject following hydrolysis of the linker.

In another embodiment, the ocular tissue is vitreous humor, aqueous humor, sub-tenon, cornea, conjunctiva, retina, choroid, or combinations thereof, preferably, the ocular tissue is vitreous humor.

In another embodiment, the hydrolysis of the linker in ocular tissues is controlled and the time for linker hydrolysis of half of the compound is selected from 1-60 minutes, 1-24 hours, or 1-30 days, preferably, 1-5 days.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 shows an illustration of how an exemplary ocular antibody-drug conjugate technology can be used to treat ocular neovascular diseases such as wet AMD.

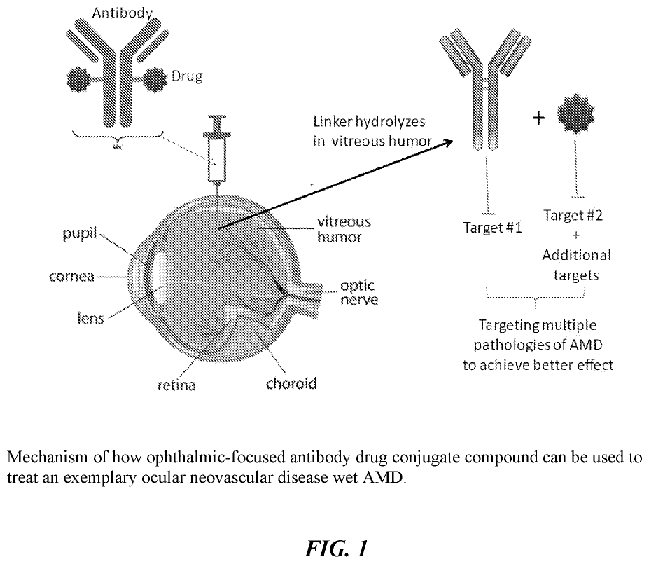

### DETAILED DESCRIPTION OF THE ILLUSTRATED EMBODIMENTS

Reference will now be made in detail to embodiments of the present invention, example of which is illustrated in the accompanying drawings.

The ocular antibody-drug conjugate disclosed here utilizes three new classes of small molecule drugs not used in the previous patent. In some embodiments, the compounds and methods described herein can be used to treat ocular diseases, such as ocular neovascular diseases. Ocular neovascular diseases are diseases of the eye that involve abnormal angiogenesis (blood vessel growth) and vessel leakage. non-limiting examples of ocular neovascular diseases include exudative (wet) and non-exudative (dry) age-related macular degeneration (AMD), diabetic macular edema, retinal vein occlusion, diabetic retinopathy, cornea neovascularization, neovascular glaucoma, adjunctive therapy for glaucoma surgery, adjunctive therapy for cornea transplant and pterygium.

Anti-angiogenesis strategies can be useful treatments for ocular neovascular diseases such as exudative AMD (also known as wet AMD). Currently, several VEGF-neutralizing biologic drugs are on the market, including anti-VEGF-A antibodies, such as bevacizumab (AVASTIN®), and ranibizumab (LUCENTIS®). These two antibody drugs are administered intravitreally about once every month. A fusion protein between VEGFR2 extracellular binding domains and antibody Fc regions, aflibercept (EYLEA®), can also be administered for the treatment of wet AMD but can be used less frequently than ranibizumab. More recently, additional biologics with similar mechanisms, brolucizumab and conbercept, have come to the market.

Despite the success of the ant-VEGF biologic drugs, there are still unmet needs for treatment of ocular neovascular diseases such as wet AMD. Some patients are resistant to anti-VEGF-A treatment. Many patients who are initially responsive can become resistant to anti-VEGF-A therapy over time. Another unmet need is to reduce the treatment burden and injection related complications. Since AMD is a complex disease with multiple pathogenic etiology, one potential way to improve treatment is to explore new disease targets in addition to the VEGF pathway or target multiple pathways at the same time.

The compounds and method described herein is a novel way to provide multiple therapeutics simultaneously to ocular neovascular diseases. In some cases, the compounds and methods described herein can increase the effectiveness of treatment, including increased effectiveness over singular treatments, or multiple unlinked therapeutics. The advantages of the compounds and methods described herein can include: 1) inhibiting more than one key disease mechanisms with a single drug molecule and a single injection; 2) increasing the effectiveness on retinal fluid removal than either single mechanism alone can achieve; 3) reducing frequency of development of resistance to a monotherapy; 4) novel route for sustained delivery of a small molecule drug to the vitreous; 5) reducing adverse effects due to frequent intravitreal injections.

The compounds and methods described herein can, in some embodiments, also be used in non-ocular tissues. For example, the antibody-drug conjugates described herein can be designed for use in treatment of non-ocular diseases, including autoimmune diseases, joint diseases, skin diseases, blood disorders, bone loss, and the like.

The compounds described herein can include an antibody or engineer biologic molecule that blocks a target, for example, a target in a subject. The antibody in the disclosed compounds and methods can be a classic antibody, an antibody hybrid fusion or any other biologic molecules that are designed to block any angiogenesis related targets. For example, in some embodiments, the antibody or engineered biologic molecule can be designed to block, without limitation, VEGF, VEGFR, PDGF, PDGFR, FGF and FGFR. Non-limiting examples of such antibodies or biologic drugs include: bevacizumab and ranibizumab, brolucizumab, aflibercept and conbercept. In addition, any anti-angiogenesis protein drugs (for example, in clinical testing but not yet approved by FDA, or newly discovered) can also be included. Non-limiting examples include anti-VEGF, -PDGF Darpins (Allergan), Sevacizumab (anti-VEGF, Jiangsu Simcere Pharmaceutical), TK001 (anti-VEGF, Jiangsu T-Mab Biopharma), Tanibirumab (anti-VEGFR2, PharmAbcine), LMG324 (anti-VEGF, Alcon/Norvatis), BCD-021 (bevacizumab biosimilar, Biocad), IMC-3G3 (anti-PDGFR, ImClone LLC), MEDI-575 (anti-PDGFR, Medimmune LLC), TRC105 (anti-endoglin antibody, NCI), Fovista (anti-PDGF, Ophthotech) and any others that inhibit VEGF, PDGF, VEGFR or PDGFR. In some embodiments, the antibody in the disclosed methods can be mono-target or bi-target or multi-target biologics. In some embodiments, the compounds described herein can be used to treat non-ocular diseases. In some embodiments, the antibody or engineered biologic molecule can be a BAFF inhibitor, an anti-CD20 antibody, a RANKL inhibitor, an IL-12 antagonist, and IL-23 antagonist, an IL-1 antagonist, an IL-1 beta antagonist, a TNF inhibitor, a TNF alpha inhibitor, a complement inhibitor, a complement C5 inhibitor, an IL-6 receptor inhibitor, an inhibitor of cell adhesion molecule α4-integrin, a T cell modulator, a CD11a binding agent or blocker, an anti-IgE antibody, a competitive antagonist of IL-2, glycoprotein IIb/IIIa receptor antagonist, or combinations thereof. In some embodiments, the antibody or engineered biologic molecule can be selected from bevacizumab, ranibizumab, brolucizumab, aflibercept, conbercept, abciximab, adalimumab, basiliximab, belimumab, canakinumab, certolizumab or certolizumab pegol, denosumab, eculizumab, efalizumab, golimumab, infliximab, natalizumab, omalizumab, tocilizumab, ustekinumab, or combinations thereof. In addition, in some embodiments, the antibody in the disclosed methods can be PEGylated.

The compounds described herein include a small drug molecule conjugated to the biologic large molecule. The small molecule can be an anti-inflammatory small molecule selected from a steroid or a NSAID, or an adrenergic receptor alpha agonist. Non-limiting exemplary steroids include dexamethasone, betamethasone, prednisone, prednisolone, triamcinolone, tethylprednisolone, hydrocortisone, cortisone acetate, fludrocortisone, aldosterone. Non-limiting exemplary NSAIDs include bromfenac, aspirin, celecoxib, diclofenac, diflunisal, etodolac, ibuprofen, indomethacin, ketoprofen, ketorolac, nabumetone, naproxen, oxaprozin, piroxicam, salsalate, sulindac, tolmetin. Non-limiting exemplary alpha agonists include apraclonidine, mivaZerol, clonidine, brimonidine, alpha methyl dopa, guanfacine, dexemeditomidine, (+)-(S)-4-1-(2,3-dimethyl-phenyl)-ethyl-1,3-dihydro-imidazole-2-thione, 1-(imidazolidin-2-yl)iminolindazole, methoxamine, phenylephrine, tizanidine, xylazine, guanabenz, amitraz.

The compounds described herein include a linker. The linker in the disclosed compounds and methods can, in some embodiments, be any kind that can be cleaved in ocular tissues and ocular cells, such as vitreous humor, aqueous humor, sub-tenon, cornea, conjunctiva, choroid, or combinations thereof. Non-limiting exemplary linkers hydrolyzable in ocular tissues or ocular cells, and other tissues, include an ester, an amide, a carbamate, a carbonate, an imine, an ether, a phosphate, a hydrazone, an acetal, or a hydrozone bond. Linkers used in the traditional ADC platforms can also be used if they can be hydrolyzed in the ocular environment. Non-limiting examples can include hydrazone, disulfide, dipeptide, beta-glucuronide). In some embodiments, the linkers may be selected to cleave in other target tissues, such as joint tissue, muscular tissue, blood, skin, epithelial tissue, connective tissue, nervous tissue, and the like. In addition, the linker can include small molecule polymer conjugate, such as PEG in the small molecule complex.

The rate of hydrolysis of the linker can be designed to be fast with the hydrolysis half-life between 1-60 minutes, or 1-24 hours. It can also be designed to be slow with half-life between 1-30 days.

The ocular antibody-drug conjugate can be delivered via intravitreal injection, subconjunctival injection, subtenon, topical eye drop or other ways to deliver to either the back or front of the eye for treating various ocular neovascular diseases. The release rate of the small molecule agent could be determined based on the course of disease progression.

Some advantages of the compounds and methods described herein can include: 1) can avoid the side effects of systemic steroid treatment by using a local delivery route; 2) the biologic drug not only can have its own efficacy against the neovascular disease but can also act as a carrier of steroid for inflammation reduction; 3) a cleavable linker can be designed to be hydrolyzed near the target tissue such as in vitreous humor, aqueous humor, sub-tenon, cornea, conjunctiva or choroid, retina within several hours to several months to prolong treatment duration, depending on the desired treatment duration, determinable by a skilled physician or other skilled artisan; 4) the compounds and methods described herein can, in some cases, allow selection of any combinations of biologic agents and small molecule agents that had proven efficacy in the clinic by themselves to achieve enhanced synergistic effects, thus enhancing the likelihood of success. Such ocular antibody-drug conjugate will enhance the effectiveness by targeting multiple pathogenic pathways of ocular neovascular diseases.

The compounds and methods described herein can be useful for treating various ocular diseases. In some embodiments, the compounds and methods described herein can be useful in treating various ocular angiogenesis and inflammatory diseases by delivering the compounds described herein to a target tissue of a subject having an ocular disease. Non-limiting exemplary ocular angiogenesis and inflammatory diseases include age-related macular degeneration (AMD), wet AMD, choroidal neovascularization (CNV), choroidal neovascular membrane (CNVM), cystoid macular edema (CME), epi-retinal membrane (ERM) and macular hole, myopia-associated choroidal neovascularisation, vascular streaks, retinal detachment, diabetic retinopathy, diabetic macular edema (DME), atrophic changes of the retinal pigment epithelium (RPE), hypertrophic changes of the retinal pigment epithelium (RPE), retinal vein occlusion, choroidal retinal vein occlusion, macular edema, macular edema due to retinal vein occlusion, retinitis pigmentosa, Stargardt's disease, glaucoma, neovascular glaucoma, adjunctive therapy for glaucoma surgery, inflammatory conditions, cataract, regractory anomalies, ceratoconus, retinopathy of prematurity, subretinal edema and intraretinal edema, angiogenesis in the front of the eye, corneal angiogenesis following keratitis, corneal transplantation or keratoplasty, corneal angiogenesis due to hypoxia and pterygium. In some embodiments, the compounds described herein can be delivered into a target ocular tissue of a subject, such as vitreous humor, aqueous humor, sub-tenon, cornea, conjunctiva, choroid, or combinations thereof. In some embodiments, the compounds described herein can be delivered into a subject's eye by topical ocular delivery or injection into intravitreal, intracameral, suprachoroidal, subconjunctival, subtenon tissue, or combinations thereof.

In some embodiments, the compounds and methods described herein can be useful for treating various non-ocular diseases by delivering the compounds described herein to a target tissue of a subject having a non-ocular disease. Non-limiting examples of non-ocular diseases that can be treated by some embodiments of the compounds and methods described herein include autoimmune diseases, joint diseases, skin diseases, blood disorders, bone loss, and the like. For example, in some embodiments, the compounds and methods described herein can be used to treat conditions such as rheumatoid arthritis, multiple sclerosis, systemic lupus erythematosus, osteoporosis, Crohn's disease, ulcerative colitis, systemic juvenile idiopathic arthritis, Still's disease, psoriatic arthritis, ankylosing spondylitis, paroxysmal nocturnal hemoglobinuria, atypical hemolytic uremic syndrome, neuromyelitis optica, psoriasis, allergic asthma, chronic spontaneous urticaria, Behcet's disease, lichen planus, transplant rejection, and the like. In some embodiments, the compounds described herein can be delivered into a target tissue of a subject, such as joint tissue, muscular tissue, blood, skin, epithelial tissue, connective tissue, nervous tissue, and the like. In some embodiments, the compounds described herein can be delivered into a subject by topical application, such as topical dermal application or mucosal application; or by injection, such as intra-articular injection, peri-articular injection, intra-muscular injection, intra-venous injection, intra-dermal injection, or sub-cutaneous injection.

Bevacizumab is linked to Dexamethasone by a linker that hydrolyzes in vitreous humor with a cleavage half-life of 2 to 15 days. Upon intravitreal injection into the vitreous humor, the bevacizumab-dexamethasone conjugate (BDC) will slowly release dexamethasone in the eye of the subject to maintain an effective concentration over the duration of the antibody's presence. Thus, the BDC compound will reduce angiogenesis and inflammation at the same time to treat an ocular disease. When wet AMD is treated by this compound, the patient will see the improved efficacy, be much less likely to develop resistance to the therapy and the treatment frequency will be reduced

### EXAMPLES

Chemicals and Lab Techniques

The chemicals were purchased from Fisher Scientific (Schwerte, Germany), Sigma Aldrich (Darmstadt, Germany), TCI (Eschborn, Germany), Broadpharm (San Diego, California USA), and Quanta BioDesign (Columbus, Ohio USA), and were used as received without further purification, unless mentioned otherwise. For TLC analysis, TLC Silica gel 60 F254 plates provided by Sigma Aldrich (Darmstadt, Germany) were used. Iodide provided Sigma Aldrich was utilized to stain the substances containing double bonds, aqueous potassium permanganate and cerium disulfate solutions were utilized to stain all oxidizable substances. Ninhydrin solution was utilized for the specific staining of amine-containing molecules. The absorption of ultraviolet light (UV) was visualized with the help of a NU-8 230 V 50 Hz 0.18 Amp UV Hand Lamp (254 nm+365 nm, 8 Watt Tube) provided by Herolab GmbH Laborgerite (Wiesloch, Germany). For silica column chromatography, Normasil 60® 40-63 μm silica gel and sand (sulphuric acid washed) provided by VWR (Dresden, Germany) was used in combination with Celite® 503 provided by Sigma Aldrich (Darmstadt, Germany).

Analytical Methods

For high pressure liquid chromatography (HPLC), an e2695 Separation Module, a 2998 Photodiode Array (PDA) Detector, a 2424 Evaporative Light Scattering (ELS) Detector, and a XBridge® Peptide BEH C18 (300 Å, 5 μm) column provided by the Waters Corporation (Milford, Massachusetts USA) were utilized in combination with Empower® 3 HPLC software which was also provided by the Waters Corporation.

For ¹H analysis, a 600 MHz Avance III system with an unshielded 52 mm bore magnet provided by Bruker (Billerica, Massachusetts USA) was utilized. The chemical shifts were listed in parts per million (ppm), and refer to the solvent residual peaks as internal standards. The NMR data reported include: chemical shifts (s: singlet, d: doublet, t: triplet, q: quartet, m: multiplet), integration, and coupling constants (s) in Hertz (Hz). Multiplets were reported over the range (in ppm) at which they appeared in the spectra. Deuterated chloroform (CDCl3) provided by Deutero GmbH (Kastellaun, Germany) was applied as solvent, unless mentioned otherwise.

For liquid chromatography-mass spectrometry (LC-MS), the samples were analyzed using an Agilent 1100 microHPLC system interfaced to an Orbitrap Velos mass spectrometer via a HESI-II ion source. The samples (0.1 μl) were injected and eluted using a short RP-HPLC gradient from 20%-90% acetonitrile. Spectra were recorded at a nominal resolution of R=60,000.

General scheme for the synthesis the compound of the present application is shown below:

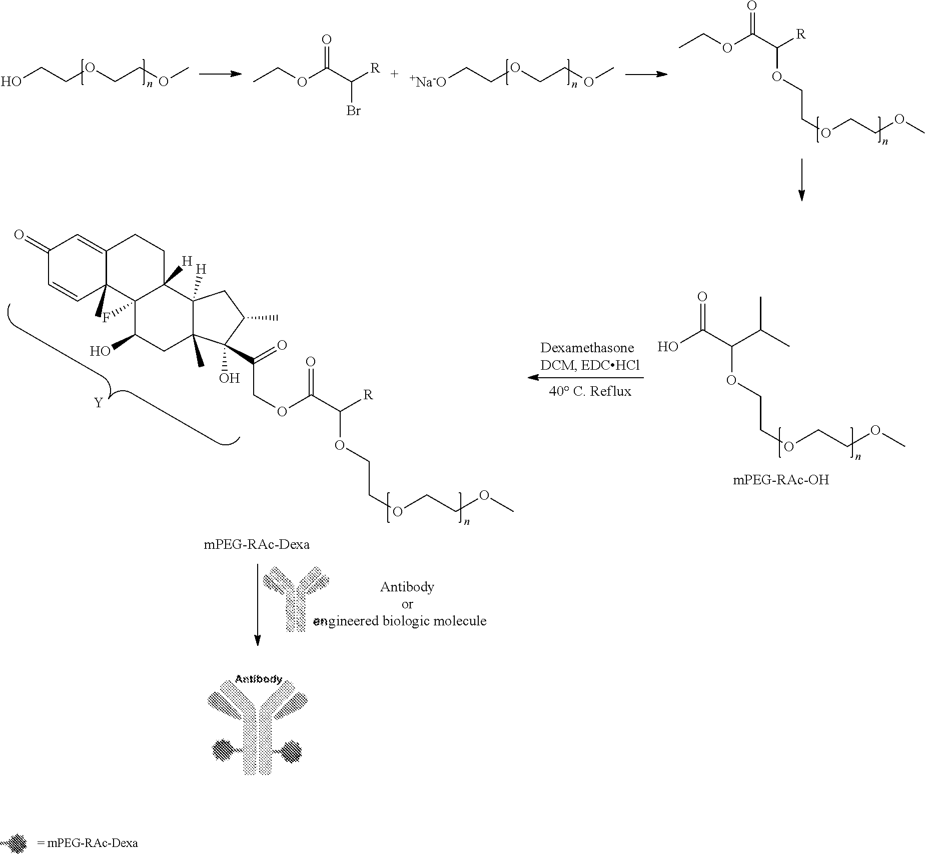

R is H, —C₁₋₁₈ alkyl, -aryl, heteroaryl, —C₁₋₁₈ alkylaryl, or -alkylheteroaryl, n is 5-30, preferably, 10-15.

mPEG-RAc-Dexa is coupled to the antibody or engineered biologic molecule via a couple reaction. For example, mPEG-RAc-Dexa can be first coupled with maleimide, and the reactive thiol of cysteine in the antibody or engineered biologic molecule can be used for coupling maleimide-containing mPEG-RAc-Dexa to the antibody or engineered biologic molecule. Other coupling methods can also be used.

### Example 1. Synthesis of mPEG-iVal-Dexa (Compound 1)

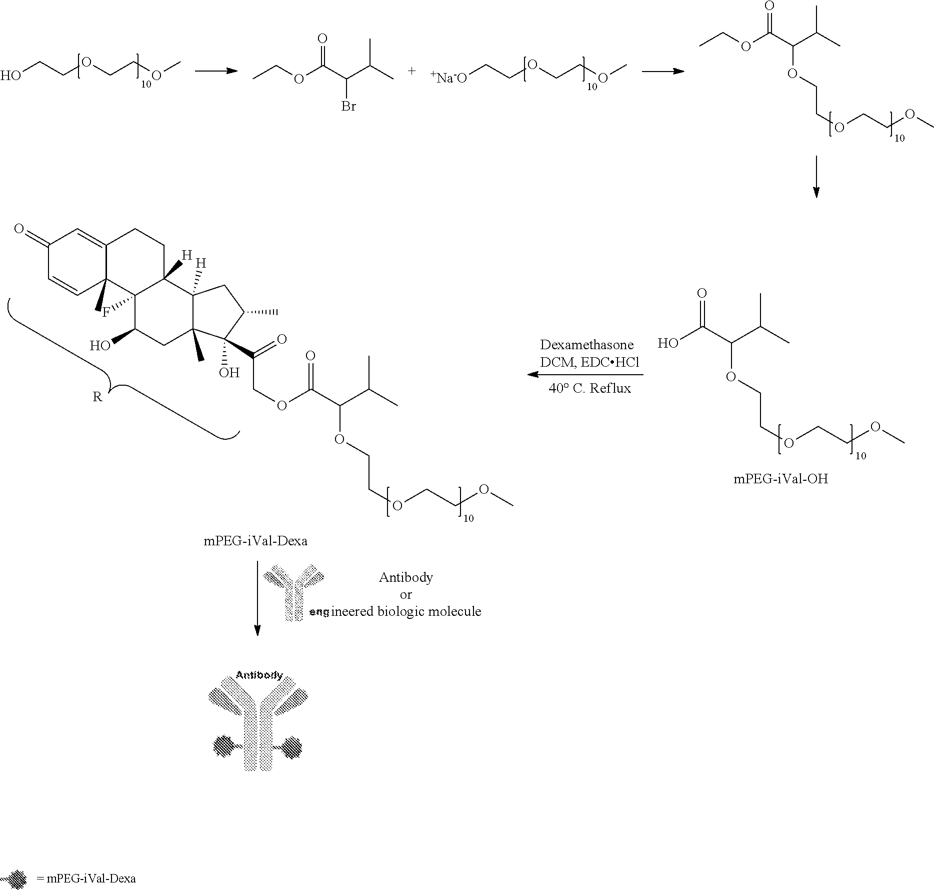

Synthesis of mPEG-iVal-OH

Sodium hydride (NaH, 60% in oil) (24 mg, 0.60 mmol, 3.16 eq.) was added to a two-neck flask. Subsequently, dimethylacetamide (DMAc) (0.5 ml) was added under stirring leading to the formation of a suspension. mPEG-OH (100 mg, 0.19 mmol, 1.0 eq.) was added to a glass vial and was dissolved in DMAc (0.7 ml). Next, the mPEG-OH solution was slowly added to the NaH suspension with the help of a syringe at room temperature. The two-neck flask was rinsed with additional DMAc (0.3 ml), ensuring that all of the reactant was added to the reaction mixture. Ethyl 2-bromoisovalerate (81 mg, 0.39 mmol, 2.0 eq.) was added to a glass vial, and was diluted with 0.5 ml DMAc. Subsequently, the ethyl 2-bromoisovalerate solution was added slowly to the reaction mixture with the help of a syringe, turning the previously clear reaction mixture slightly yellow over time. Analysis by means of HPLC after 18 h of reaction time at room temperature showed no conversion of the starting materials. Therefore additional ethyl 2-bromoisovalerate (100 mg, 0.48 mmol, 2.5 eq.) was added to the reaction mixture. Analysis by means of HPLC showed no conversion after 2 h of additional reaction time. On that account, some additional NaH (spatula scoup) was added to the reaction mixture. The formation of H₂ gas was observed, indicating that a reaction took place. After 1 h, analysis by means of HPLC did not show any remaining ethyl 2-bromoisovalerate. Subsequently, additional ethyl 2-bromoisovalerate (80 mg, 0.38 mmol, 2.0 eq.), an additional scoup of NaH, and some additional DMAc (0.5 ml) were added to the reaction mixture, and the resulting reaction mixture was stirred for 19 h at room temperature. The reaction was then quenched with EtOH (1 ml) and the reaction mixture was diluted with H2O (2 ml)+0.2 M NaOH (aq) (1.0 ml). The resulting reaction mixture was stirred at 40° C. for 18 h (i.e., in order to hydrolyse the ethyl ester). Next, the reaction mixture was transferred to a separatory funnel, and the reaction mixture was washed with tert-butyl methyl ether (2×10 ml). The water phase was acidified with the help of 1 M HCl solution (1.2 ml). Subsequently, the water phase was extracted with DCM (3×10 ml). The combined DCM layers were then washed with 0.1 M citric acid solution (3×10 ml). Thereafter, the solvent was removed with the help of a rotary evaporator system and the obtained product was characterized by means of proton nuclear magnetic resonance spectroscopy (1H NMR), and high pressure liquid chromatography (HPLC) with ELSD monitoring. ¹H NMR [600 MHz, δ (ppm), CDCl₃]: 4.26 (d, J=7.2 Hz, 1H), 3.61 (m, 2H), 3.51 (m, 42H), 3.44-3.37 (m, 4H), 3.25 (s, 3H), 2.11 (m, 1H), 1.01 (d, J=7.2 Hz, 3H), 0.98 (d, J=6.6 Hz, 3H).

Synthesis of mPEG-iVal-Dexa

Dexamethasone (21.4 mg, 3.37×10-2 mmol, 1.0 eq), 1-Ethyl-3-(3-dimethylaminopropyl) carbodiimide-hydrochloride (EDC·HCl) (14.0 mg, 7.30×10-2 mmol, 1.3 eq), and 4-Dimethylamino pyridine (DMAP) (9.5 mg, 7.78×10-2 mmol, 1.3 eq) were suspended in dichloromethane (DCM) (1 ml). Subsequently, a solution of mPEG-iVal-OH prepared above (45 mg, 7.09×10-2 mmol, 1.3 eq) in DCM (1 ml) was added to the suspension. Next, the flask was rinsed with an additional milliliter of DCM. The reaction mixture was stirred under reflux at 40° C. under the exclusion of air (i.e., under nitrogen atmosphere). The in-process-control of the reaction was performed by means of HPLC with ELSD monitoring. After HPLC did confirm the disappearance of the starting material, the reaction mixture was washed in a separatory funnel with 2×3 ml 0.1 M citric acid (pH 2.0). Subsequently, the organic layer was washed with 2×3 ml 0.15 M NaHCO₃ (aq) (pH 8-9). Finally, the organic layer was washed with 1×5 ml deionized H2O, after which it was dried with the help of anhydrous MgSO₄. The solvent was removed under reduced pressure, utilizing a rotary evaporator, and the crude was obtained. Next, the crude was purified by column chromatography using 4 v % acetone in DCM as the eluent. The purified product was analyzed by means of ¹H NMR, COSY NMR, LC-MC, and HPLC with ELSD monitoring. ¹H NMR [600 MHz, δ (ppm), DMSO-d6]: 7.29 (dd, J=1.2 Hz, 10.2 Hz, 1H, Dexa), 6.23 (dd, 1.8 Hz, 10.2 Hz, 1H, Dexa), 6.01 (s, 1H, Dexa), 5.41 (ddd, J=1.2 Hz, 5.4 Hz, 16.8 Hz, 1H, Dexa), 4.15 (m, 1H, Dexa), 3.84 (d, J=4.8 Hz, 0.5H, Dexa), 3.80 (d, J=5.4 Hz, 0.5H, Dexa), 3.69 (m, 1H, mPEG-iVal), 3.51 (m, 34H, mPEG-iVal), 3.43 (m, 2H, mPEG-iVal), 3.24 (s, 3H, mPEG-iVal), 1.25 (m, 1H, Dexa), 2.88 (m, 1H, Dexa), 2.61 (dt, J=5.4 Hz, 13.8 Hz, 1H, Dexa), 2.42-2.28 (m, 2H, Dexa), 1.77 (m, 1.19H, Dexa), 1.69-1.55 (m, 2H, Dexa), 1.49 (s, 3H, Dexa), 1.08 (m, 1H, Dexa), 0.79 (dd, J=1.8 Hz, 7.8 Hz, 3H, Dexa).

### Example 2: Synthesis of mPEG-PrA-Dexa (Compound 2)

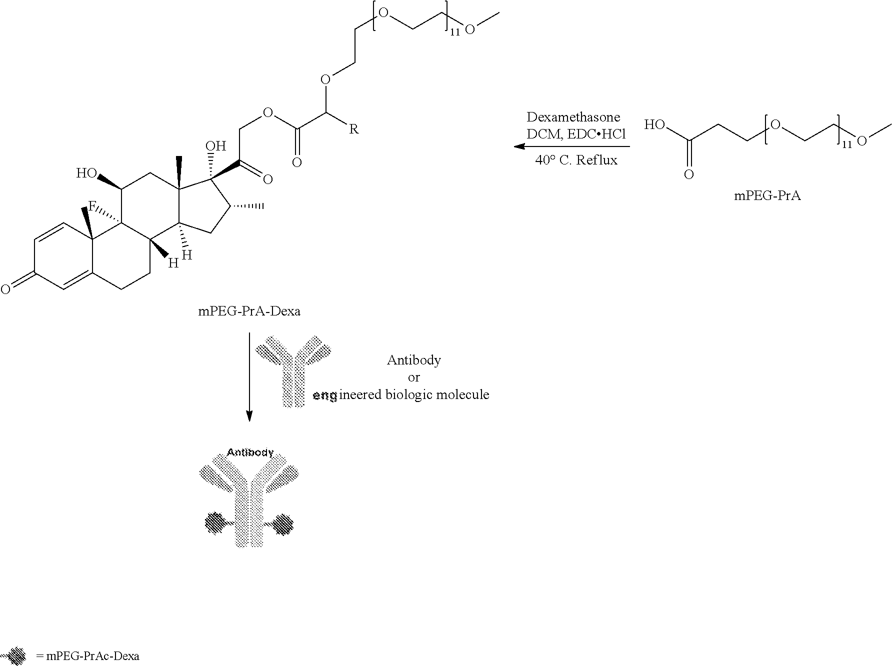

mPEG₁₂-COOH (195 mg, 0.33 mmol, 1.3 eq) was dissolved in dry dichloromethane (DCM) (40 ml). Subsequently, dexamethasone (100 mg, 0.26 mmol, 1.0 eq.), 4-dimethylaminopyridine (40 mg, 0.33 mmol, 1.3 eq.), and 1-Ethyl-3-(3-dimethylaminopropyl)carbodiimide-hydrochloride (EDC·HCl) (63 mg, 0.33 mmol, 1.3 eq.) were added to the solution. The reaction was stirred under reflux at 40° C. for 10 hours. Thin layer chromatography was utilized to confirm the formation of the product (mobile phase: 50 v % Acetone in DCM with iodide as coloring agent). The product was purified by means of column chromatography, utilizing the same mobile phase as for TLC. The purified fractions were combined, and the solvent was removed under reduced pressure using a rotary evaporator. The purified product was characterized by means of proton nuclear magnetic resonance spectroscopy (¹H NMR), high pressure liquid chromatography (HPLC), and liquid chromatography-mass spectrometry (LC-MS). ¹H NMR [600 MHz, δ (ppm), CDCl₃]: 7.21 (d, 1=10.2 Hz, 1H, Dexa), 6.33 (dd, J=1.8 Hz, 10.2 Hz, 1H, Dexa), 6.11 (s, 1H, Dexa), 4.90 (m, 2H, Dexa), 4.37 (d, J=9.6 Hz, 1H, Dexa), 3.79 (m, 2H, mPEG-Acid), 3.67-3.60 (m, 40H, mPEG-Acid), 3.55 (m, 2H, mPEG-Acid), 3.38 (s, 3H, mPEG-Acid), 3.09 (m, 1H, Dexa), 2.73 (t, J=6.6 Hz, 2H, Dexa), 2.61 (dt, J=5.4 Hz, 12.0 Hz, 1H, mPEG-Acid), 2.45-2.33 (m, 3H, Dexa), 2.25 (s, 1H, Dexa), 2.18 (m, 2H, Dexa), 1.82 (m, 1H, Dexa), 1.76 (q, J=12 Hz, 1H, Dexa), 1.66 (s, 3H, Dexa), 1.25 (m, 1H, Dexa), 1.22 (m, 1H, Dexa), 1.05 (s, 3H, Dexa), 0.93 (d, J=7.8 Hz, 3H, Dexa).

### Example 3: Synthesis of mPEG-tertBuG-Dexa (Compound 3)

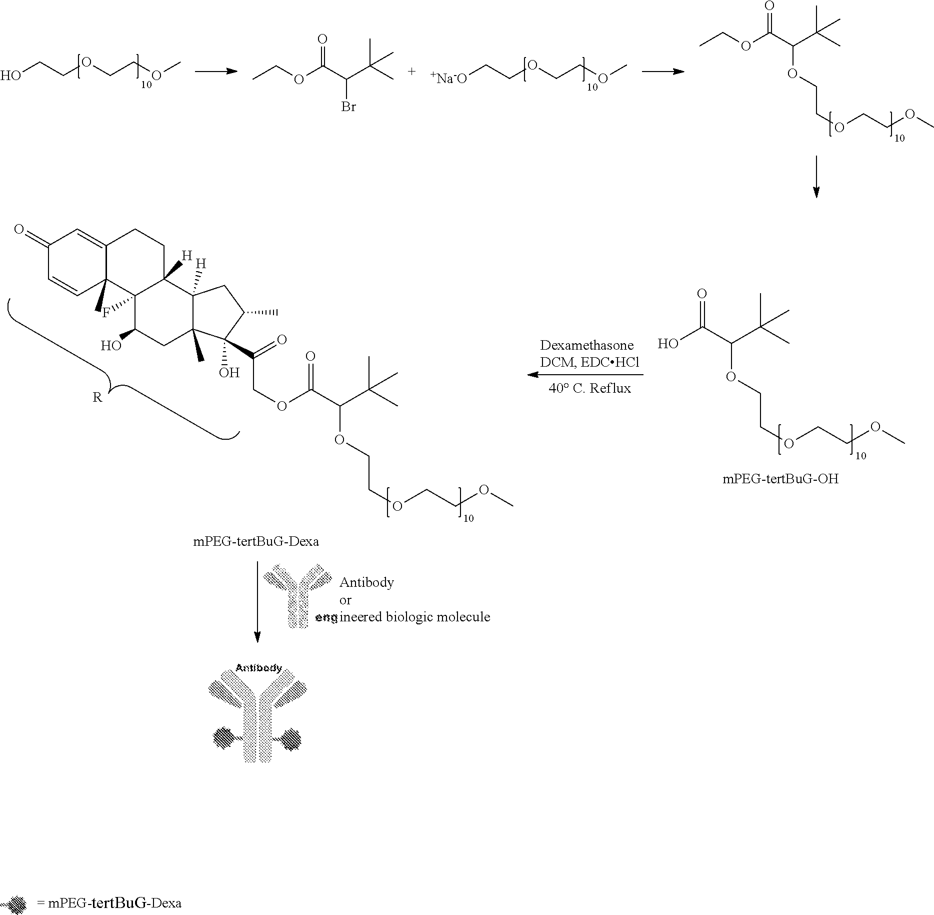

Compound 3 was synthesized using the chemistry similar to the synthesis of compound 1, shown in the above scheme. ¹H NMR [600 MHz, δ (ppm), DMSO-d6]: 7.29 (dd, J=1.2 Hz, 10.2 Hz, 1H, Dexa), 6.23 (dd, 1.8 Hz, 10.2 Hz, 1H, Dexa), 6.01 (s, 1H, Dexa), 5.41 (ddd, J=1.2 Hz, 5.4 Hz, 16.8 Hz, 1H, Dexa), 4.15 (m, 1H, Dexa), 3.84 (d, J=4.8 Hz, 0.5H, Dexa), 3.81 (s, 1H, mPEG-tertBuG), 3.80 (d, J=5.4 Hz, 0.5H, Dexa), 3.51 (m, 34H, mPEG-tertBuG), 3.43 (m, 2H, mPEG-tertBuG), 3.24 (s, 3H, mPEG-tertBuG), 1.25 (m, 1H, Dexa), 2.88 (m, 1H, Dexa), 2.61 (dt, J=5.4 Hz, 13.8 Hz, 1H, Dexa), 2.42-2.28 (m, 2H, Dexa), 1.77 (m, 1.19H, Dexa), 1.69-1.55 (m, 2H, Dexa), 1.49 (s, 3H, Dexa), 1.08 (m, 1H, Dexa), 0.89 (s, 9H, tertBuG), 0.79 (dd, J=1.8 Hz, 7.8 Hz, 3H, Dexa).

### Example 4: Synthesis of mPEG-HCA-Dexa (Compound 4)

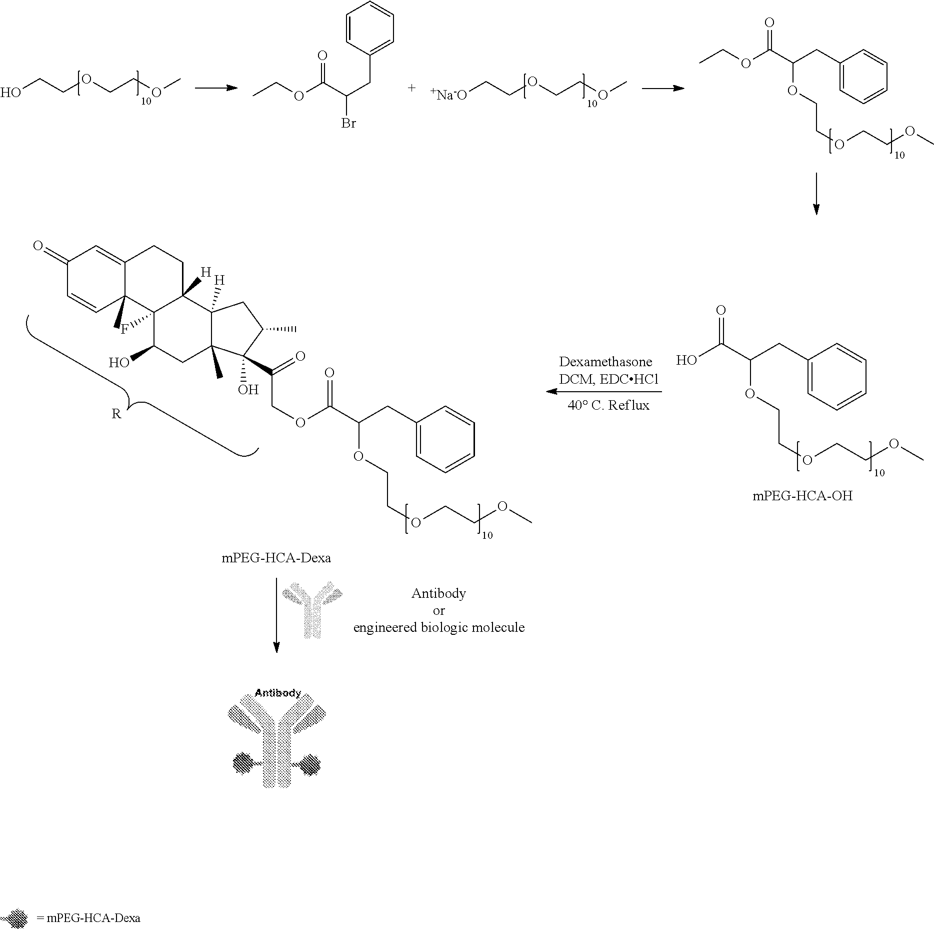

Compound 4 was synthesized using the chemistry similar to the synthesis of compound 1, shown in the above scheme. ¹H NMR [600 MHz, δ (ppm), DMSO-d6]: 7.29 (dd, J=1.2 Hz, 10.2 Hz, 1H, Dexa), 7.19-7.23 (m, 5H, mPEG-HCA), 6.23 (dd, 1.8 Hz, 10.2 Hz, 1H, Dexa), 6.01 (s, 1H, Dexa), 4.51 (d, J=7.0 Hz, 1H, mPEG-HCA), 5.41 (ddd, J=1.2 Hz, 5.4 Hz, 16.8 Hz, 1H, Dexa), 4.15 (m, 1H, Dexa), 3.84 (d, J=4.8 Hz, 0.5H, Dexa), 3.80 (d, J=5.4 Hz, 0.5H, Dexa), 3.51 (m, 34H, mPEG-HCA), 3.07 (dd, 2H, J=1.8, 7.0 Hz, mPEG-HCA), 1.25 (m, 1H, Dexa), 2.88 (m, 1H, Dexa), 2.61 (dt, J=5.4 Hz, 13.8 Hz, 1H, Dexa), 2.42-2.28 (m, 2H, Dexa), 1.77 (m, 1.19H, Dexa), 1.69-1.55 (m, 2H, Dexa), 1.49 (s, 3H, Dexa), 1.08 (m, 1H, Dexa), 0.79 (dd, J=1.8 Hz, 7.8 Hz, 3H, Dexa).

### Example 5: Selective and Controlled Hydrolysis of Compounds 1 and 2 In Vitreous Humor

Vitreous Humor Homogenate Preparation: vitreous humor homogenate was prepared from New Zealand rabbits. After extraction from rabbit eyes, vitreous humor was transferred to cold, pre-weighed centrifuge tubes with screw caps, and maintained at −80° C. The vitreous humor was thawed in 50 mL centrifuge tube, added with 5 mL of 0.1% sodium diethyl-dithiol-carbamate for every 100 mL of vitreous humor. Some small beans were added and stirred into the jelly state vitreous humor at 0° C. until viscosity was reduced to close to water. The protein concentration was determined with UV absorption and was diluted to a final concentration of 10.0 mg/ml and was then centrifuged at 2500 rpm for 15 min at 4° C. The supernatant was collected for testing hydrolysis of conjugates.

Hydrolysis assessment of Compounds 1 and 2: 198 μL vitreous humor homogenate or phosphate buffer was added to each tube and gently mixed. The 2 μL working solution of either the test compound or the control compound was added and mixed. The samples were incubated at 37° C. and the reaction was stopped by adding 200 μL of quenching solution at 0, 1, 4, 8, 12, 24, 48 and 72 and vortexed vigorously for −1 min. The solution was then centrifuged at 4,000 rpm at 4° C. for 15 min. The 100 μL of the supernatant was removed and mixed with 100 μL distilled water for LC-MS/MS analysis. The 10 μL standard curve working solution was spiked to 190 μL phosphate solution, The 200 μL of quenching solution was added to each standard curve wells. The blank sample was prepared by adding 2 μL of MeOH/DMSO solvent to replace the working solution. The double blank samples was prepared by adding 2 μL of MeOH/DMSO solvent to replace the working solution and by quenching the matrix with acetonitrile/MeOH. The concentration of Compounds 1 and 2 (parent compounds) and the concentration of dexamethasone (released from parents by hydrolysis) were analyzed with LC-MS/MS.

Hydrolysis rate of Compounds 1 and 2: the hydrolysis and release of dexamethasone from Compounds 1 and 2 were analyzed in vitreous humor in an in vitro study and the results are shown in Table 1 below. Hydrolysis was observed in vitreous humor, not in the water control. The half-lives of hydrolysis and dexamethasone release were about 96 and 17 hours for Compounds 1 and 2, respectively (Table 1). The results supported our invention of selective and controlled hydrolysis of the conjugates in vitreous humor to have sustained effects of both the small drug and antibody drug. 1) The linkage can be selectively hydrolyzed in vitreous humor; 2) the hydrolysis rate can be tuned by adding bulky groups to the linker, exemplified by Compound 1 vs Compound 2, to increase the half-life of hydrolysis.

TABLE 1
Half-lives of hydrolysis of example compounds
and release of dexamethasone in vitreous humor

| | T ½ in vitreous humor | T ½ in water control |
|---|---|---|
| Compound 1 | 95.7 hours | No hydrolysis |
| Compound 2 | 17.2 hours | No hydrolysis |

### REFERENCES

JoN, Mailhos C, Ju M, Cheung E, Bradley J, Nishijima K, Robinson G S, Adamis A P, and Shima D T. Inhibition of Platelet-Derived Growth Factor B Signaling Enhances the Efficacy of Anti-Vascular Endothelial Growth Factor Therapy in Multiple Models of Ocular Neovascularization. Am J Path. 2006; 168: 2037-53.
Kim E G and Kim K M. Strategies and Advancement in Antibody-Drug Conjugate Optimization for Targeted Cancer Therapeutics. Biomol Ther. 2015; 23(6): 493-509.
Martin D F, Maguire M G, Ying G S et al. Ranibizumab and bevacizumab for neovascular age-related macular degeneration. The CATT Research Group. N. Engl. J. Med. 2011; 364(20): 1897-1908.
Peters C. and Brown S. Antibody-drug conjugates as novel anti-cancer Chemotherapeutics. Bioscience Reports. 2015; 35: e00225.
Rosenfeld P J, Brown D M, Heier J S et al. Ranibizumab for neovascular age-related macular degeneration. N. Engl. J. Med. 2006; 355(14): 1419-1431.
Stewart M W, Grippon S, Kirkpatrick P. Aflibercept. Nat. Rev. Drug Discov. 2012; 11(4): 269-270.
Yang S, Zhao J, Sun X. Resistance to anti-VEGF therapy in neovascular age-related macular degeneration: a comprehensive review. Drug Des Devel Ther. 2016 Jun. 2; 10:1857-67.

1. A compound comprising:
an antibody or engineered biologic molecule that blocks VEGF, VEGFR, PDGF, PDGFR, FGF, or FGFR;
a small molecule drug, the small molecular drug being an adrenergic receptor alpha agonist or an anti-inflammatory small molecule, the anti-inflammatory small molecule being a steroid or a non-steroid anti-inflammatory drug (NSAID); and
a linker that links the small molecule drug to the antibody or engineered biologic molecule,
wherein the linker comprises a bond that can be hydrolyzed in ocular tissue in a controlled release fashion.

2. The compound of claim 1, wherein the antibody is an anti-VEGF-A antibody.

3. The compound of claim 1, wherein the antibody is selected from group consisting of bevacizumab, ranibizumab, brolucizumab, aflibercept, and conbercept.

4. The compound of claim 1, wherein the antibody is pegylated to include a polyethylene glycol (PEG) moiety that is either linear or branched.

5. The compound of claim 4, wherein the PEG moiety is —(CH₂—CH₂—O—)ₙ—, and n is 5-30.

6. The compound of claim 1, wherein the linker links the small molecule drug via the PEG moiety to the antibody or engineered biologic molecule.

7. The compound of claim 1, wherein the linker comprises an ester, an amide, a carbamate, a carbonate, an imine, an ether, a phosphate, a hydrazone, an acetal, or a hydrozone bond, preferably, the linker comprises an ester bond.

8. The compound of claim 7, wherein the linker is 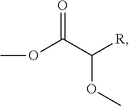
wherein R is H, —C1-18 alkyl, -aryl, heteroaryl, —C1-18 alkylaryl, or -alkylheteroaryl, preferably, R is H, methyl, ethyl, propyl, isopropyl, t-butyl, phenyl, or benzyl.

9. The compound of claim 1, wherein the steroid is selected from group consisting of dexamethasone, betamethasone, prednisone, prednisolone, triamcinolone, tethylprednisolone, hydrocortisone, cortisone acetate, fludrocortisone, and aldosterone, preferably, the steroid is dexamethasone.

10. The compound of claim 1, wherein the NSAID is selected from the group consisting of aspirin, celecoxib, bromfenac, diclofenac, diflunisal, etodolac, ibuprofen, indomethacin, ketoprofen, ketorolac, nabumetone, naproxen, oxaprozin, piroxicam, salsalate, sulindac, and tolmetin, preferably, the NSAID is bromfenac.

11. The compound of claim 1, wherein the adrenergic receptor alpha agonist is selected from the group consisting of apraclonidine, mivaZerol, clonidine, brimonidine, alpha methyl dopa, guanfacine, dexemeditomidine, (+)-(S)-4-1-(2,3-dimethyl-phenyl)-ethyl-1,3-dihydro-imidazole-2-thione, 1-(imidazolidin-2-yl)iminolindazole, methoxamine, phenylephrine, tizanidine, xylazine, guanabenz, and amitraz, preferably, the adrenergic receptor alpha agonist is brimonidine.

12. The compound of claim 1, wherein the compound comprises:
bevacizumab; dexamethasone; a polyethylene glycol (PEG) moiety; and a linker,
wherein the PEG moiety is —(CH2-CH2-O-)n-, n is 5-30;
wherein the linker is 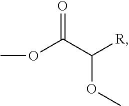
and R is H, methyl, ethyl, propyl, isopropyl, t-butyl, phenyl, or benzyl; and
wherein the linker links dexamethasone via the PEG moiety to bevacizumab.

13. The compound of claim 1, wherein the hydrolysis of the linker in ocular tissues is controlled and a time for linker hydrolysis of a half of the compound is 1-60 minutes, 1-24 hours, 1-5 days, or 1-30 days, preferably, 1-5 days.

14. A method of treating an ocular disease in a subject, comprising:
delivering the compound of claim 1 to an eye of the subject.

15. The method of claim 14, further comprising:
allowing the linker to hydrolyze in one or more ocular tissues of the eye of the subject over time; wherein both the antibody and the small molecule drug exert their functions in the subject following hydrolysis of the linker.

16. The method of claim 15, wherein the ocular tissue is vitreous humor, aqueous humor, sub-tenon, cornea, conjunctiva, retina, choroid, or combinations thereof, preferably, the ocular tissue is vitreous humor.

17. The method of claim 14, wherein the hydrolysis of the linker in ocular tissues is controlled and the time for linker hydrolysis of half of the compound is selected from 1-60 minutes, 1-24 hours, or 1-30 days, preferably, 1-5 days.

18. The compound of claim 3, wherein the antibody is bevacizumab.

19. The compound of claim 5, wherein n is 10-15.
